(19) United States
(12) Patent Application Publication
(10) Pub. No.: US 2009/0317293 A1
(43) Pub. Date: Dec. 24, 2009

(54) METHOD AND DEVICE FOR ELIMINATING MICROBES WITHIN INDUSTRIAL PIPELINES

(75) Inventors: Cale Street, Edmonds, WA (US); Joe Ridge, Seattle, WA (US); Nicolas Loebel, Redmond, WA (US); Roger Andersen, Ladysmith (CA)

Correspondence Address: DOBRUSIN & THENNISCH PC, 29 W LAWRENCE ST, SUITE 210, PONTIAC, MI, 48342, US

(73) Assignee: BioCorrosion Solutions Inc.

(21) Appl. No.: 12/431,991

(22) Filed: Apr. 29, 2009

Related U.S. Application Data
(60) Provisional application No. 61/048,991, filed on Jun. 18, 2008.

(51) Int. Cl. A61L 2/18; A61L 2/02

(52) U.S. Cl. 422/28; 422/1; 422/243; 422/292

(57) ABSTRACT

The present invention provides a method to inhibit microbes located within a pipeline comprising: applying a photosensitizer composition to a predetermined interior surface area of the pipeline where microbes are located; and applying light to the area at a wavelength that is absorbed by the photosensitizer composition so as to inhibit or eliminate the microbes; wherein the light is applied by a photodynamic disinfection device adapted to provide light to the area. The present invention also provided a photodynamic disinfection device comprising a pipeline inspection gauge (pig) adapted to include a circumferential high energy light source and an energy source wherein the light source is in communication with and powered by the energy source via communication means.

### CLAIM OF BENEFIT OF FILING DATE

This application claims the benefit of U.S. Provisional Application Ser. No. 61/048,991 titled: “Method and Device for Eliminating Microbes Within Pipelines” filed on Jun. 18, 2008 and PCT Application No. PCT/US09/41928 titled: “Method and Device for Eliminating Microbes Within Pipelines” filed on Apr. 28, 2009.

### FIELD OF THE INVENTION

The present invention relates to a method and a device to inhibit and/or eliminate microbes within industrial pipelines and processing systems.

### BACKGROUND OF THE INVENTION

Microbial growth on the interior surface of industrial pipelines and processing systems is a common problem, especially in the worldwide oil and gas industries. Bacteria that are either natively resident in oil and gas or introduced after the extraction process adhere to the interior surface of the pipelines and processing systems, forming a biofilm ultrastructure on such surface. These biofilms, which are very durable and resistant to physical removal, are generally formed when colonizing microbes encapsulate themselves together in a slimy, exopolymeric substance composed of secreted polysaccharides, proteins, and nucleic acids. Biofilms are much more difficult to eradicate by conventional means (biocides, physical/mechanical scraping) than planktonic bacteria due to strong adherence to surfaces and physical exclusion of antimicrobial substances. Once a local biofilm has been established in a pipeline, metabolic activity of the organisms produces organic acids and gases that are harmful to carbon steel. In addition, some types of bacteria are able to metabolize sulfur and release by-products that are highly corrosive to steel. A 2003 study by Zhu et al. used advanced molecular techniques to characterize the microbial communities harvested from standard gas pipelines. A total of 106 different bacterial DNA sequences were identified in these samples, and among those identified were species that produce nitrates (contribute to metal corrosion), species that are known contributors to biofilm formation, organic acid producers, sulfate reducers, and hydrogen consuming methanogens. The damage to the pipeline structure that results from the action of these bacteria has been termed microbiologically-influenced corrosion (MIC). For example, around forty percent (40%) of the internal pipeline corrosion in the gas industry has been attributed to MIC. MIC also accounts for up to twenty-five percent (25%) of pipeline failure incidents. When pipeline failures occur the effects on the environment, workers safety, and product revenues are nothing short of disastrous. MIC is a multi-billion dollar problem in the industrial pipeline industry worldwide.

Microbes and related biofilm also occur in other industrial pipelines and processing systems (e.g., water treatment, etc.). The threat of excessive microbial growth along the interior surface of drinking water distribution pipelines is a health concern. The development of biofilm within such industrial pipelines and processing systems is also commonly known as biofouling. It is present in almost every water distribution system, and when uncontrolled may present a threat to public health. The biofilm can clog water lines to the point of insufficient water pressure. In addition, biofilm contributes to further pipe corrosion and can deplete chlorine used to disinfect drinking water and maintain water quality.

It is well known that the microbes in biofilm behave differently (genetically and physiologically) from their planktonic counterparts. Furthermore, the microbes in biofilm are much more difficult to eradicate by conventional means (e.g., traditional biocides, physical/mechanical scraping, etc.) than planktonic microbes because of their strong adherence to surfaces and physical exclusion of antimicrobial substances.

A large proportion of existing pipeline infrastructure exists in remote geographical areas. Furthermore, it is challenging to access the inside of existing lines making internal corrosion difficult to address. Pipeline inspection gauges (“pigs”) are devices that are commonly used in the pipeline industry for cleaning, eliminating blockage, separating product, and electronic mapping within difficult to access lines. There are many different types of pigs utilized for different purposes, including mandrel pigs, foam pigs, and gel pigs. In addition, “smart” pigs are used that have onboard electronics for telemetry and data collection.

Photodynamic disinfection is a technology used in the biomedical field for the treatment of bacterial infections in conditions such as periodontitis. This technology fundamentally involves the use of light energy to activate one or more photosensitizers of a photosensitizing composition so that those photosensitizers can then either pass energy on directly to a substrate/target (type I reaction), or can interact with molecular oxygen to produce singlet oxygen or other oxygen-derived free radicals (type II reaction). These reactions mediate the non-specific killing of microbial cells primarily via lipid peroxidation, membrane damage, and damage to intracellular components.

### SUMMARY OF THE INVENTION

A large proportion of existing pipeline infrastructure exists in remote geographical areas. Furthermore, it is challenging to access the inside of existing lines making internal corrosion difficult to address. Pipeline inspection gauges (“pigs”) are devices that are commonly used in the pipeline industry for cleaning, eliminating blockage, separating product, and electronic mapping within difficult to access lines. There are many different types of pigs utilized for different purposes, including mandrel pigs, foam pigs, and gel pigs. In addition, “smart” pigs are used that have onboard electronics for telemetry and data collection.

Photodynamic disinfection is a technology used in the biomedical field for the treatment of bacterial infections in conditions such as periodontitis. This technology fundamentally involves the use of light energy to activate one or more photosensitizers of a photosensitizing composition so that those photosensitizers can then either pass energy on directly to a substrate/target (type I reaction), or can interact with molecular oxygen to produce singlet oxygen or other oxygen-derived free radicals (type II reaction). These reactions mediate the non-specific killing of microbial cells primarily via lipid peroxidation, membrane damage, and damage to intracellular components. Deployed in industrial applications, major advantages of photodynamic disinfection would include the fact that it has broad spectrum activity against most known types of bacteria (including those in biofilm form), the ability to eradicate high percentages (usually >99%) of bacterial populations, and relatively small environmental impact compared to traditional chemical agents used as industrial biocides. Photodynamic disinfection deployed on the interior surface of industrial pipelines and processing systems (collectively hereinafter referred to as “pipelines”), is significantly different from the application of this technology in the biomedical field. This is because the insides of pipelines are largely anaerobic environments (i.e. no oxygen present) with greatly different types of bacteria from those that associate with living tissues. For these reasons, the past application of photodynamic disinfection for biomedical applications does not suggest nor anticipate that the transfer of the technology to pipelines.

The present invention provides a powerful and yet relatively environmentally-safe method, based on photodynamic disinfection, for inhibiting and/or eliminating microbes (in both planktonic and biofilm forms) residing within pipelines. This method does not cause structural damage to the pipelines. The method includes applying a photosensitizer composition to a predetermined interior surface area of a pipeline where microbes are located and applying light to the area at a wavelength that is absorbed by the photosensitizer composition so as to inhibit or eliminate the microbes, wherein the light is applied by a light delivery device adapted to provide light to the area.

The present invention also presents a pipeline photodynamic disinfection device comprising: a pipeline inspection gauge (“pig”) adapted to include a circumferential high-energy light source and an energy source wherein the light source is in communication with and powered by the energy source. The device of the present invention may optionally further include a cartridge segment adapted to contain and to dispense the photosensitizing composition and a reservoir for collecting used photosensitizer composition.

There are currently pigs used in industrial application that are equipped with onboard light sources. For example, Smart Light Devices, Ltd. located in Aberdeen, Scotland, U.K. (www.sldltd.com) produces a smart pig that is equipped with an onboard laser which projects ahead of the pig as it travels for the purpose of scanning and 3-D internal pipeline imaging. There are several key differences between current pigs that use onboard light for scanning/visualization and the photodynamic disinfection pig contemplated in the present invention. Firstly, the light energy doses necessary at the pipeline surface for effective photodynamic disinfection are much greater than those achieved during visualization processes. Second, this energy must be delivered over a greater surface area circumferentially within the pipe during photodynamic disinfection, as opposed to scanning lasers, which only project a very thin beam of light along the inner pipeline wall. Finally, the light wavelengths from the photodynamic disinfection pig are chosen to photochemically activate a photosensitizer composition in order to eradicate bacteria, as opposed to other onboard light sources which are used for measurement purposes.

### DETAILED DESCRIPTION OF THE DRAWINGS

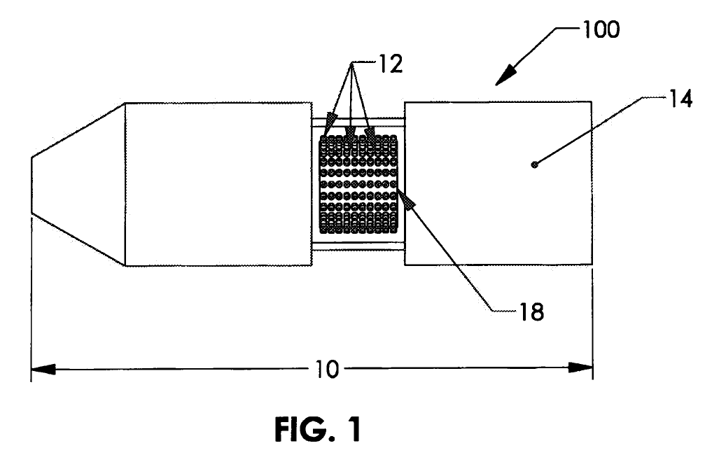

FIG. 1 is a side view of an exemplary embodiment of a pipeline photodynamic disinfection pig device according to the present invention;

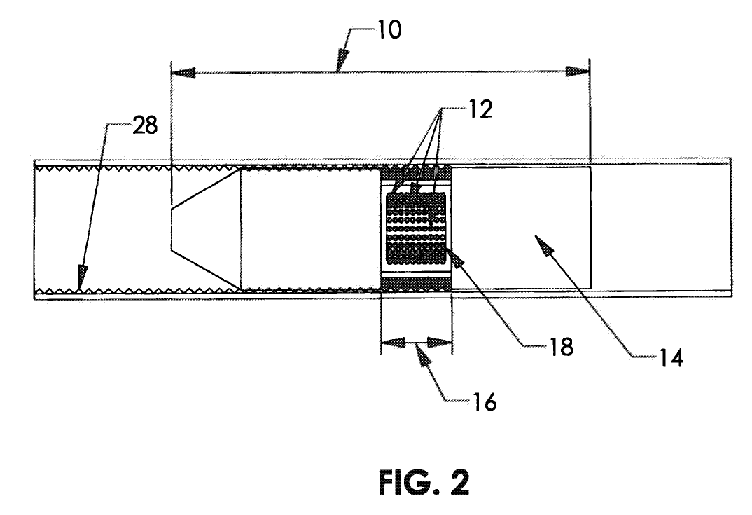

FIG. 2 is a side view of the device shown in FIG. 1 when it is located within internal cavity of a pipeline during the application of the photodynamic disinfection method according to the present invention;

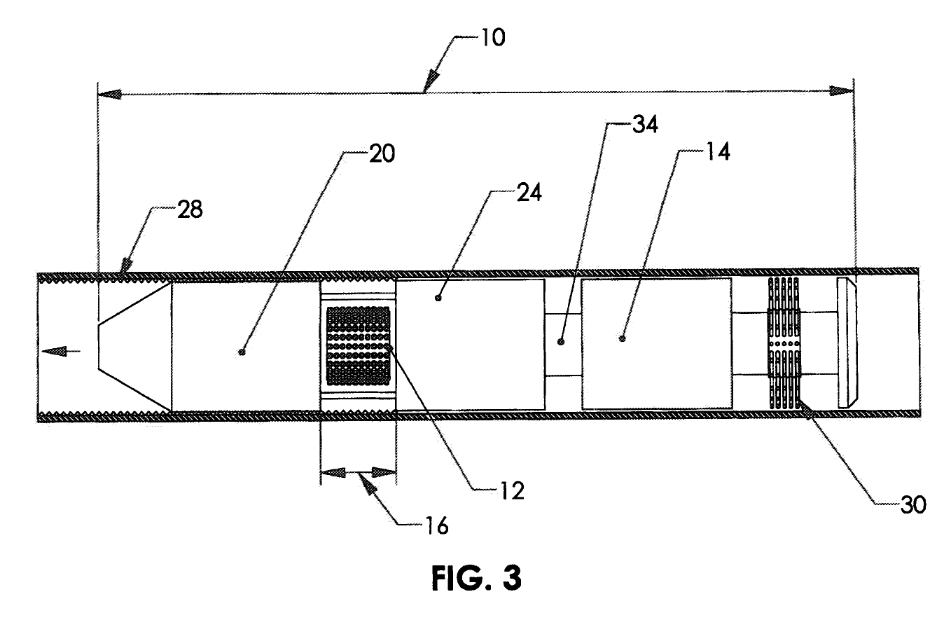

FIG. 3 is an isometric view of another exemplary embodiment of a pipeline photodynamic disinfection device according to the present invention;

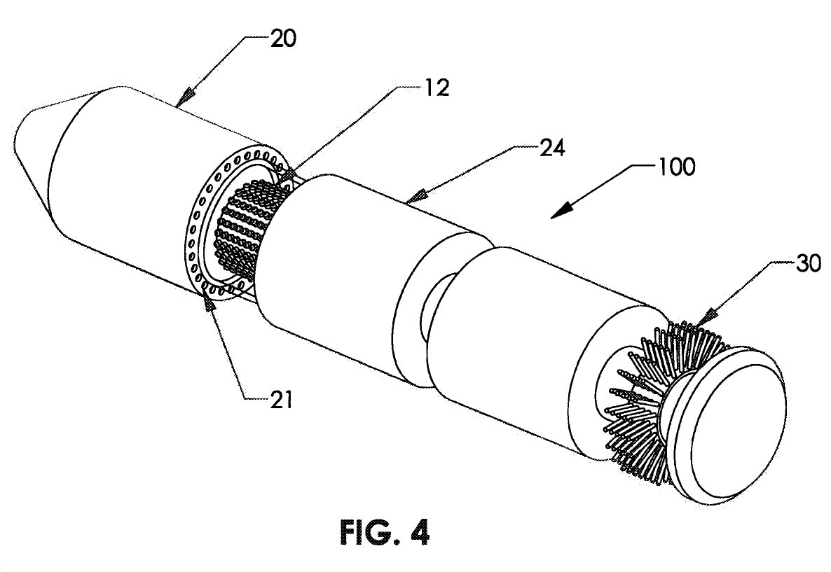

FIG. 4 is a side view of the device shown in FIG. 3 when it is located within internal cavity of a pipeline during the application of the photodynamic disinfection method according to the present invention;

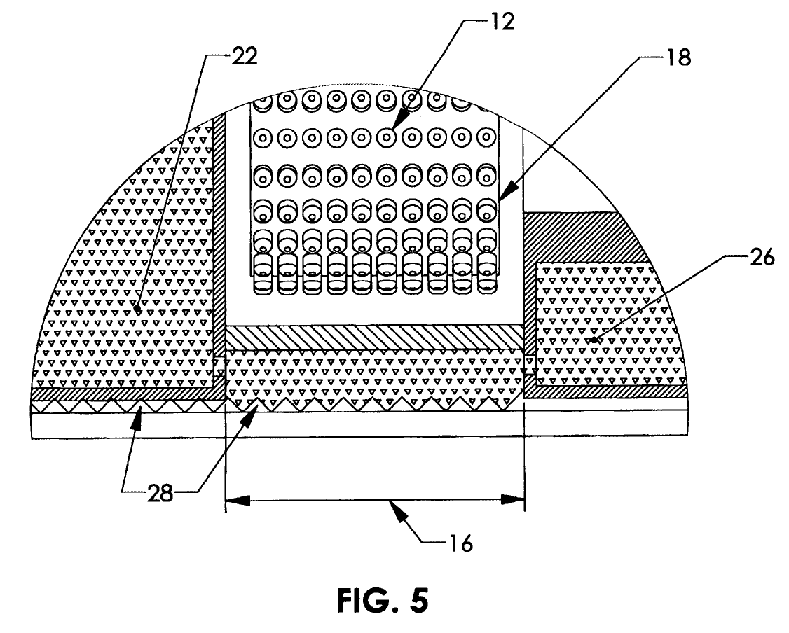

FIG. 5 is a cross section view of a portion of the device shown in FIG. 3 during the application of the photodynamic disinfection method according to the present invention;

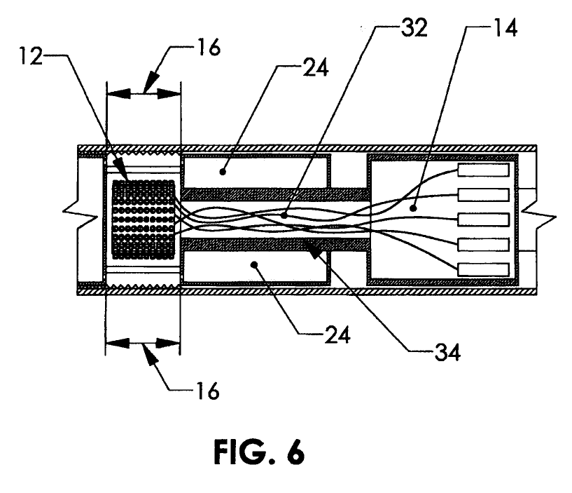

FIG. 6 is another cross section view of a portion of the device shown in FIG. 3; and

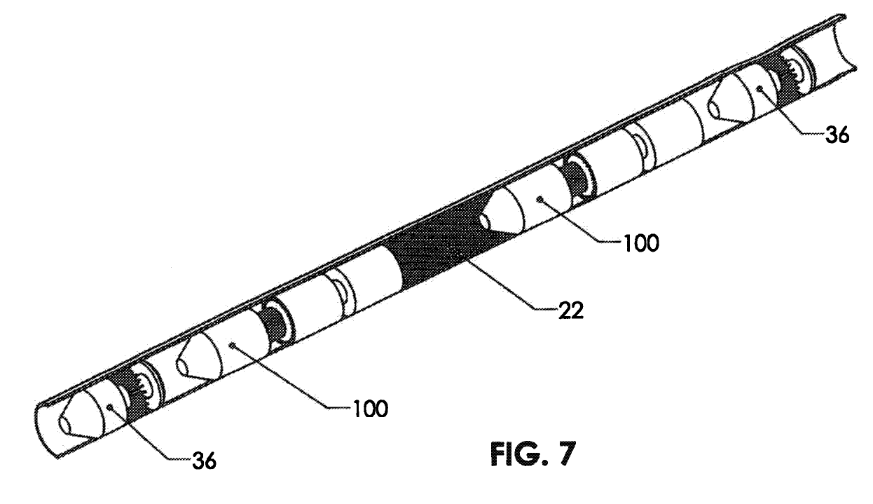

FIG. 7 is an isometric view of two cleaning pigs and two photodynamic disinfection devices of the present invention within internal cavity of a pipeline during the application of the photodynamic disinfection method according to the present invention.

### DESCRIPTION OF THE PREFERRED EMBODIMENT

### I. Definitions

The following terms are intended to have the following general meanings as they are used herein:

1. Light: light at any wavelengths that can be absorbed by a photosensitizer composition. Such wavelengths include wavelengths selected from the continuous electromagnetic spectrum such as ultraviolet (“UV”), visible, the infrared (near, mid and far), etc. The wavelengths are generally between about 100 nm to 10,000 nm, with exemplary ranges between about 160 nm to 1600 nm, between about 400 nm to about 900 nm, and between about 500 nm to about 850 nm, although the wavelengths may vary depending upon the particular photosensitizing compound used and the light intensity. Depending on the application, the light produced may be in a very narrow (i.e. <10 nm) or broad (up to 100's of nm) wavelength range.

2. Circumferential High Energy Light Source: a light source comprising of suitable art-disclosed light emitting device(s) wherein the light source is adapted, arranged and/or designed to provide illumination in a circumferential fashion. Examples of the suitable art-disclosed light emitting device(s) are laser(s), light emitting diode(s) (“LED(s)”), incandescent source(s), fluorescent source(s), arc lamp source(s) (e.g., neon, argon, xenon, krypton, sodium, metal halide, mercury, or the like), or a combination thereof. An example of the circumferential high energy light source is an array of LEDs adapted and designed to provide illumination in a circumferential fashion. The output of the light source is optionally adjustable so that the operator can modify the wavelength, the power output, the surface area of illumination, or combinations thereof while carrying out the present method. Alternately, the power of the light source may be increased or decreased after an application of light energy to the pipeline area.

3. Microbes: any and all biofilm-related microbes capable of colonizing and causing damage within a pipeline. Examples of microbes that generally colonize and cause damage to pipelines in the gas and oil industries are Enterobacter and Citrobacter bacteria (e.g., E. dissolvens, E. ludwigii, C. farmeri and C. amalonaticus, etc.); Eubacterium and Clostridium bacteria (e.g., Clostridium butyricum, Clostridium algidixylanolyticum, Anaeorfilum pentosovorans, Bacteroides sp., Acinebacter sp., Propionibacterium sp., etc.); sulfate reducing bacteria including but not limited to Desulfovibrionales (e.g., Desulfovibrio desulfuricans, Desulfovibrio vulgaris, Desulfovibrio aminophilus, etc.); Desulfobacterales, and Syntrophobacterales; thiosulfate reducing anaerobes (e.g., Geotoga aestuarianis, Halanaerobium congolense, Sulfurospirillum sp., etc.); tetracholoroethene degrading anaerobes (e.g., Sporomusa ovata, etc.); triethanolamine degrading bacteria (e.g., Acetobacterium sp., etc.); denitrifiers (e.g., Acidovorax sp., Pseudomonas sp., etc.); xylan degrading bacteria; Nitrospirae; Halomonas spp.; Idiomarina spp.; Marinobacter aquaeolei; Thalassospira sp.; Silicibacter sp.; Chromohalobacter sp.; Bacilli (e.g., Bacillus spp. Exiguobacterium spp., etc.); Comamonas denitrificans; Methanobacteriales; Methanomicrobiales; Methanosarcinales; etc. Examples of microbes that generally colonize and cause damage to pipelines in other industries are: Staphylococcus aureus, Methicillin-resistant Staphylococcus aureus (“MRSA”), Escherichia coli, Enterococcus fecalis, Pseudomonas aeruginosa, Aspergillus, Candida, Clostridium difficile, Staphylococcus epidermidis, Acinobacter sp., etc.

4. Photosensitizer composition: a composition comprising at least one suitable art-disclosed photosensitizer that has at least an antimicrobial action upon application of electromagnetic energy of certain wavelength(s). Suitable photosensitizers include those that act via one or both of Type I and Type II photoreaction antimicrobial mechanisms, where Type I involves the excited-state photosensitizer molecule engaging in direct redox-type reactions with the target substrate, and Type II involves the interaction of excited-state photosensitizer with molecular oxygen to produce singlet oxygen and other damaging oxygen-derived species. When applied to microbes, both processes lead to irreversible protein damage, lipid peroxidation, and loss of membrane integrity. While photosensitizers may exert an antimicrobial effect via other mechanisms (e.g. heat generation and thermal mechanisms), the Type I and II mechanisms discussed above are preferred.

Suitable classes of compounds that may be used as antimicrobial photosensitizers include tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; 6,607,522 and 7,276,494. Preferred phenothiazines include methylene blue (MB), new methylene blue (NMB), dimethyl methylene blue (DMMB), toluidine blue (TBO), and those discussed in U.S. Patent Publication No. 2004-0147508. Other preferred antimicrobial photosensitizers include indocyanine green (ICG), safranine compounds (safranin-O), and rose bengal. Combinations of two or more photosensitizers, such as MB and TBO or the like, are also suitable. The photosensitizers mentioned above are examples and are not intended to limit the scope of the present invention in any way.

The photosensitizer may be present in the photosensitizer composition in any suitable amounts. Examples are between about 0.001 percentage of total weight (wt %) and 50 wt %, between about 0.005 wt % and about 25 wt %, between about 0.01 wt % to about 10 wt %, between about 0.01 wt % to about 5 wt %, between about 0.1% wt % to about 1 wt %.

The photosensitizer composition may also optionally include carriers, diluents, excipients, or other solvents for the photosensitizer or other components of the photosensitizer composition. In addition, the photosensitizer molecule of the photosensitizer composition may be chemically/physically conjugated or otherwise mixed with targeting moieties (including but not limited to peptides, phages, or antibodies) or enhancers of photodynamic disinfection efficacy. Finally, the photosensitizer composition may optionally include other bactericides/bacteriostats (e.g., chlorhexidine, benzalkonium chloride, glutaraldehyde, antibiotics etc.) that use a different mechanism(s) of antimicrobial action.

5. Inhibit: The term “inhibit” shall mean disinfect, inhibit, damage, eliminate, reduce, and/or kill.

### II. Pipeline Photodynamic Disinfection Method

The present invention provides a method to inhibit and/or eliminate microbes located within a pipeline comprising: applying a photosensitizer composition to a predetermined interior surface area of the pipeline (i.e., a desired treatment area) where microbes are located. The photosensitizer composition is preferred to have a viscosity that is flowable but still can effectively adhere onto the interior surface of the pipeline. The photosensitizer composition can be manufactured in a liquid form or in a non-liquid form that becomes flowable when placed into the pipeline. In one example, the photosensitizer composition may initially have a lower viscosity and then increase in its viscosity (e.g., gel, foams, or the like.) after it has entered into the pipeline, thereby permitting a more effective adherence onto interior surface of the pipeline. In another example, the liquid photosensitizer composition may be sprayed at high velocity onto the biofilm/pipeline surface, using a suitable dispersion device. One example of such a dispersion device is the V-jet™ inhibitor dispersal system created by T.D. Williamson Inc. located in Tulsa, Okla., USA (www.tdwilliamson.com). In one alternative embodiment, the method may optionally further include applying an art-disclosed oxygen enhancing compound into the photosensitizer composition before application or into the pipeline at the time of treatment to further increase antimicrobial efficacy. The oxygen enhancing compound can increase free radical/singlet oxygen yield or prolong singlet oxygen half-life. Examples of such oxygen enhancing compound include: 1) dissolved atmospheric oxygen (“O₂”), 2) oxygen carrying molecules capable of releasing oxygen or increasing the partial pressure of oxygen in liquid solution (e.g., hemoglobin, perfluorochemical based compounds), or 3) molecules that can undergo reactions to yield reactive oxygen species (alcoholic oxygens, ethereal oxygens, H₂O₂, superoxide, etc.).

The application of the oxygen enhancing compound can be simultaneous or sequential to the application of the photosensitizer composition. For example, if the oxygen enhancing compound is in gaseous form, it can be delivered to the treatment area via compressed gas canisters fitted to the photodynamic disinfection device discussed below. Alternatively, the oxygen enhancing compound can be combined with the photosensitizer composition and apply together as one solution to the interior surface of the pipeline.

The method of the present invention further includes applying light to a predetermined interior surface area of the pipeline at a wavelength that is absorbed by the photosensitizer composition so as to inhibit the microbes, wherein the light is applied by a mobile device adapted to provide light to such area. In another embodiment of the method, the photosensitizer composition application step (i.e., applying the photosensitizing composition to a predetermined interior surface area of the pipeline where microbes are located) and the light application step (i.e., applying light to the area at a wavelength that is absorbed by the photosensitizer composition so as to inhibit or eliminate the microbes) are both achieved by the device of the present invention.

The method can be performed by (1) applying the photosensitizer composition first and then by applying the light; or (2) applying the photosensitizer composition and the light simultaneously.

The amount of time desired for the light application step (“treatment time”) depends on many factors including the nature and concentration of the photosensitizer composition, and the power density (milliwatts per square centimeter) of light energy applied. For example, the treatment time can be from about 1 second to about 5 minutes, from about 30 seconds to about 3 minutes, from about 60 seconds to about 2 minutes, or the like.

Depending on their designed purposes, conventional pipeline pigs generally travel at a broad range of speeds within a pipeline. Typical pigs used for cleaning or clearing the pipeline generally move along the internal cavity of a pipeline at the same speed as the product being transported through the line (approximately 1 to 2 meters per second). Pigs can also be slowed in the pipeline using various techniques and design features. Examples of the speed at which the photodisinfection device travels during photodisinfection treatment are at a rate about less than about 2 m per second, less than about 1 m per second, less than about 50 cm per second.

It is preferred that each cm² of the pipeline inner surface area to be treated using photodynamic disinfection receive an energy dose of at least about 0.01 Joule/cm² or greater; however the activation energy required for complete photodynamic disinfection will be highly dependent on the nature and concentration of the photosensitizer composition and the nature and extent of the microbes located at the treatment site.

Depending on the nature and extent of the microbes located at the treatment site, using multiple photodynamic disinfection devices in a train formation will allow multiple cycles of light applications to the same treatment site thereby resulting in a total accumulated light energy dose applied to the treatment site that can be substantially higher than the light energy provided during each cycle. It is preferred that selections of the photosensitizer composition, the light wavelength(s) to be applied, and/or the total cumulative light energy dose to be applied to the treatment site will allow the method of the present invention to kill over about 90%, more preferably over about 95%, and most preferably over about 99% of the targeted microbes at the treatment site. It is also preferred that the application of light to the treatment site does not cause damage to the pipeline structure and/or product(s) being transported by such pipeline.

### III. Pipeline Photodynamic Disinfection Device

Referring to FIG. 1, the present invention also provides a pipeline photodynamic disinfection device 100 comprising a pipeline inspection gauge 10 (“pig”) adapted to include a circumferential high energy light source 12 and an energy source 14 wherein the light source 12 is in communication with and powered by the energy source 14. It is optionally that the pig 10 includes means to control (remotely or otherwise) the speed of the device 100 as it travels through the pipeline.

Pigs are devices that are launched into a pipeline and propelled along the interior of the pipeline for various purposes. A pig is typically propelled along the pipeline under the flow and/or pressure of pipeline product in fluid form (i.e., gas and/or liquids). In the absence of any speed control means for the pig, it generally propels along the interior of the pipeline at the same speed as the fluid product being transported in the pipeline. Pigs were originally developed to remove deposits which could obstruct or retard product flow through a pipeline. They are currently used during all phases in the life of a pipeline for many different reasons (e.g., internal pipeline inspection; internal pipeline cleaning and/or debridement; physical separation between different liquids being transported in a pipeline; capturing and recording geometric information of a pipeline such as size; position, or the like; etc.).

There are many types of pigs (e.g., utility pigs, in line inspection pigs, or the like). In-line inspection pigs are designed to provide information about the condition of a particular section of pipeline, including the location of any problems. Utility pigs are used for physical separation between different liquids being transported in a pipeline, and for cleaning and/or debridement purposes to remove deposits and debris from a pipeline. Each of these pigs can come in a number of forms, including but not limited to: mandrel pigs, foam pigs, solid cast pigs, spherical pigs, caliper pigs, or the like. See, e.g., pigs manufactured by T.D. Williamson, Inc. (www.tdwilliamson.com/pigtdw/pipe_pig.html); Pipeline Pigging Products, Inc. located in Houston, Tex., USA (www.pipepigs.com); Sweco Fab, Inc. located in Houston, Tex., USA; Knapp Polly Pig located in Houston, Tex., USA (www.pollypig.com); U.S. Bellows, Inc., Div. of Piping Technology & Products, Inc. located in Houston, Tex., USA (www.pipingtech.com); Northtown Company located in Huntington Beach, Calif., USA (www.northtowncompany.com/pipeline.html); Piping Technology & Products, Inc. located in Houston, Tex., USA (www.pipingtech.com/products/fabricated.htm); Coastal Corrosion Control, Inc. located in Baton Rouge, La., USA (www.coastalcorrosion.com); Girard Industries located in Houston, Tex., USA; TDW Pigging Products located in Tulsa, Okla., USA; Apache Pipeline Products, located in Edmonton, Alberta, Canada (www.apachepipe.com); IST Pigging Systems located in Cincinnati, Ohio, USA; IPS International, Ltd. located in Mansfield, Tex., USA. Another example of a pig is the UltraScan® Duo manufactured by GE Energy located in Atlanta, Ga. (www.gepower.com). See also, U.S. Pat. Nos. 4,769,598, 5,208,936, 6,190,090, 6,370,721, 6,640,655, and 6,931,149 for additional examples of pigs. For example and as shown in FIGS. 1-2, a utility pig such as a mandrel pig can be designed and manufactured to include the light source 12 and the energy source 14 for applying photodynamic disinfection.

Referring again to FIG. 1 and FIG. 2, the light source 12 is optionally separated from desired treatment site 16 by any suitable art-disclosed material 18 that is transparent or translucent to the illumination wavelengths (i.e., light) that will be supplied by the light source 12. Examples of the material 18 are plastic, epoxy, glass, or the like. The illumination pattern of the light source 12 can be impacted by geometry and/or surface finish of the material 18. For example, the material 18 may include surface finishes such as smoothness, roughness, ribs, inclusions, pigments, microspheres, facets, embossed patterns, or a combination thereof to modify or enhance the illumination pattern (e.g., light scattering or the like) during photodynamic disinfection.

The light source 12 is adapted to deliver light (i.e., illumination) circumferentially to the treatment site 16 (i.e., a predetermined interior surface area of the pipeline desired for treatment) at wavelength(s) that will activate the photosensitizer composition so as to inhibit the microbes in the treatment site 16. Referring to FIG. 2, the treatment site 16 is the desired length or distance of the pipeline's interior surface area designated for each application or pulse of light applied by the light source 12 during photodynamic disinfection. The diameter of the circumferential light source 12 is less than the diameter of the internal cavity of the pipeline in order to allow the application of the photosensitizer composition and light to the treatment site 16. For example, the diameter of the light source 12 can be from about 1 mm to about 10 cm and from about 2 mm to about 4 cm less than the diameter of the internal cavity of the pipeline. The length of the treatment site 16 depends upon the illumination pattern provided by the light source 12. For example, the treatment site 16 can be from about 5 cm to about 1 m and from about 10 cm to about 50 cm in length.

The energy source 14 can be any suitable art-disclosed energy source (e.g., a battery or the like) that can provide sufficient power to operate the light source 12 in a fashion that allows the light source 12 to provide a desired illumination at a desired power density for a desired amount of treatment time without causing any structural damage to the treated pipeline itself. It is optional that a battery pack acting as the energy source 14 includes control means that allows that the energy source 14 to be controlled remotely (e.g., via radio frequency, radioisotope thermoelectric generators, or the like).

In another embodiment of the device 100 shown in FIGS. 3-6, the device 100 further includes a fitted cartridge segment 20 adapted to contain and to dispense the photosensitizer composition 22 and a reservoir segment 24 for collecting used photosensitizer composition 26. The cartridge 20 and the reservoir 24 can be constructed out of any suitable art-disclosed material that can withstand the conditions within a pipeline. Examples of such material are plastic (e.g., polycarbonate plastic or the like), epoxy, polyurethane, steel, metal alloy, or the like. The cartridge 20 and the reservoir 22 are designed to be of appropriate size and shape to fit as a component of the pig 10. In one embodiment, the cartridge 20 and/or the reservoir 24 are designed to be removable. In another embodiment, the cartridge 20 and/or the reservoir 24 are not removable but include an opening (not shown) or the like that allows filing and/or draining of fluids (e.g., the photosensitizer composition 22, the used photosensitizer composition 26, etc.).

The cartridge 20 can dispense the photosensitizer composition 22 either passively (e.g., utilizing gravity, pressure differences, or the like) or actively using suitable art-disclosed dispensing means 21 onto the treatment site 16 covered with microbes 28 (shown in their biofilm form in FIG. 5). For example, if the photosensitizer composition 22 has a relatively low viscosity, the dispensing means 21 may be a spray and pump assembly. If the photosensitizer composition 22 has a higher viscosity, the dispensing means 21 may be a nozzle and pump assembly to exude the high viscosity photosensitizing composition 22 from the upstream cartridge 20 and onto the treatment site 16. The dispensing means 21, if desired, can be powered either by the energy source 14 or a separate energy source.

The downstream reservoir 22 can collect the used photosensitizer composition 26 (i.e., the photosensitizer composition 22 after it has been applied to the treatment site 16 and activated by light provided by the light source 12) either passively or actively. For example, the used photosensitizer composition 26 can be driven passively out and away from the treatment site 16 by fluid pressure from incoming fresh photosensitizer composition 22. Alternatively, the reservoir 22 may optionally include suitable art-disclosed removal means (not shown) that facilitate the removal and delivery of the used photosensitizer composition 26 from the treatment site 16 into the reservoir 22. Examples of suitable removal means are scraping mechanisms, suction/pumping mechanisms, or the like. The removal means, if desired, can be powered by either by the energy source 14 or a separate energy source.

Referring to FIG. 5, a detailed view of a portion of the device 100 wherein the cartridge segment 20 is passively dispensing the photosensitizer composition 22 onto the treatment site 16, allowing the light source 12 to provide light onto the treatment site 16 and activate such dispensed photosensitizer composition 22, thereby killing the microbes 28 located on the treatment site 16. After such application of light, the used photosensitizer composition 26 located in the treatment site 16 is passively collected into the reservoir 24. In an effort to ensure better antimicrobial efficacy and prevent exhaustion of the photocatalytic properties of the photosensitizer composition 22, the used photosensitizer composition 26 is continuously moved away from the treatment site 16 into the reservoir 22 in order to allow fresh photosensitizer composition 22 to move in and contact the treatment site 16 as the device 100 moves along the internal cavity of the pipeline.

FIG. 6 shows an example of how the light source 12 is in energy communication with the energy source 14 via art-disclosed communication means 32 (e.g., electrical cables or the like) when the reservoir 24 is located between the light source 12 and the energy source 14. In this example, the device 100 includes an internal channel 34 between the light source 12 and the energy source 14 adapted to contain and isolate the communication means 32 from the environment. The internal channel 34 (as shown in FIGS. 3 and 6) may pass through the reservoir 24 and any other components of the pig 10 that are between the light source 12 and the energy source 14. The internal channel 32 allows the communication means 32 to pass through multiple pig components without compromising speed, travel, or access to the treatment site 16. Alternatively, the energy source 14 can be physically located adjacent to the active component(s) for which it is supplying energy to.

Referring to FIGS. 3-4, it is contemplated that the device 100 may optionally include cleaning means 30 that facilitate further mechanical removal of microbial biofilms from the treatment site 16. The cleaning means 30 can be any suitable art-disclosed cleaning devices designed for cleaning a pipeline (e.g., a wire brush assembly or the like.) If the cleaning means 30 requires energy, it can be powered by either by the energy source 14 or a separate energy source.

The device 100 may also optionally include additional cleaning means 30 that facilitate mechanical removal of microbial biofilms and/or other debris from the treatment site 16 before photodynamic disinfection. Such additional cleaning means 30 would be placed ahead of the cartridge 20 and/or the light source 12 allowing removal of microbes and/or other debris before photodynamic disinfection. Alternatively, the inner pipeline surface could be prepared for optimal photodynamic disinfection by using separate standard wire brush pigs or the like to physically remove the bulk of deposits before application of photodynamic disinfection. In this way, the photodynamic disinfection pig would be used to eradicate the remaining microbes on the pipeline surface after physical disruption, thus preventing immediate biofilm regrowth.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

The following examples provided in accordance to the present invention are for illustrative purpose only and are not intended as being exhaustive or limiting of the invention.

### Example I

The following method of the present invention to inhibit and/or eliminate microbes located within a pipeline is provided as follows. The device 100 described above and shown in FIG. 3-6 is launched into a section of an industry pipeline in need of antimicrobial treatment. The cartridge 20 of the device 100 is fully filled with the photosensitizer composition 22. The device 100 is launched according to standard pig launch procedures and driven by differential pressure and/or flow. As the device 100 travels along the pipeline, the photosensitizer composition 22 is passively dispensed from the cartridge 20 into the treatment site 16 while light at a predetermined wavelength designed to activate the photosensitizer composition is also applied to the treatment site 16 by the light source 12.

Upon application of the light, the photosensitizer composition 22 contains activated photosensitizer molecules that directly inhibit the living microbial cells within the microbial biofilm structure. The activated photosensitizer molecules also may interact with oxygen molecules that have been dispersed into the treatment site 16 to create singlet oxygen and other oxygen-derived free radicals that also inhibit the living microbial cells within the microbial biofilm structure. These photoreactions inhibit the microbes located on the treatment site 16. As the device 100 continues to travel down the pipeline, the used photosensitizer composition 26 is passively collected by the reservoir 24.

### Example II

Same as Example I wherein the photosensitizer composition is comprised of about 0.1% w/v methylene blue; the light, applied by the light source 12 comprising an LED array, has a center wavelength of about 670 nm; and the light energy dose per cycle of light applied to the treatment site 16 is at least about 1.0 Joule/cm².

### Example III

Same as Example I wherein the photosensitizer composition is comprised of a mixture of several different photosensitizer molecules, all exhibiting peak optical absorbance at different wavelengths. An example of such photosensitizer composition would include rose Bengal (lambdamax 370 nm), safranin O (lambdamax 521 nm), toluidine blue O (lambdamax 635 nm), and methylene blue (lambdamax 665 nm). The light, applied by the light source 12 comprising an array of broadband light emitters such as arc lamps, has a wide wavelength across the entire visible light range. The light energy dose per cycle of light applied to the treatment site 16 is at least 1.0 Joule/cm².

### Example IV

Same as Example I wherein the photosensitizer composition includes an oxygen carrier compound saturated with O₂ molecules.

### Example V

Same as Example I except that a mechanical brush cleaning pig is deployed ahead of the photodynamic disinfection pig device 100 to partially remove large deposits of debris and/or biofilm so that the device 100 can travel more freely and have better access to remaining microbes at the treatment site(s) 16. Alternatively, the device 100 further includes cleaning means 30 to remove microbial debris from the treatment sites 16 before photodynamic disinfection.

### Example VI

Same as Example I except that the device 100 further includes cleaning means 30 to remove microbial debris from the treatment site(s) 16 after photodynamic disinfection. Alternatively, a separate cleaning (e.g., a mechanical scraping/debridement) pig is deployed behind the device 100 to remove dead microbes and debris from the treatment site(s) 16 after photodynamic disinfection.

### Example VII

FIG. 7 illustrates another embodiment of the photodynamic disinfection method of the present invention. In this embodiment, two or more of the photodynamic disinfection devices (100) are used to form a train in order to deliver more light energy and sustained photodynamic disinfection to the treatment site(s) 16. This train can be preceded and/or followed by separate mechanical brush cleaning pigs 36 to remove microbes and other debris. Alternatively, such cleaning can be achieved by the optional cleaning means 30 to be included in each (or just some) of the photodynamic disinfection devices (100) within the train.

Each photodynamic disinfection device 100 within the train can be separated by the product that is flowing in the pipeline at the time of launch. Alternatively, each photodynamic disinfection device 100 within the train can be separated by a predetermined volume of the photosensitizer composition 22, which can coat the interior pipeline surface and penetrate the microbes between each of the photodynamic disinfection devices 100 within the train and allow for more antimicrobial efficacy.

### Example VIII

Same as Example I wherein the pipeline is an oil pipeline and the method is used to prevent and/or treat MIC by inhibiting at least one of the following microbes: Enterobacter and Citrobacter bacteria; Eubacterium and Clostridium bacteria; sulfate reducing bacteria including but not limited to Desulfovibrionales, Desulfobacterales, and Syntrophobacterales; thiosulfate reducing anaerobes; tetracholoroethene degrading anaerobes; triethanolamine degrading bacteria; denitrifiers; xylan degrading bacteria; Nitrospirae; Halomonas spp.; Idiomarina spp.; Marinobacter aquaeolei; Thalassospira sp.; Silicibacter sp.; Chromohalobacter sp.; Bacilli; Comamonas denitrificans; Methanobacteriales; Methanomicrobiales; and Methanosarcinales.

### Example IX

### Photodynamic Killing of Sulfate-Reducing Bacteria in Aerobic and Anaerobic Conditions

An in vitro photodynamic disinfection model was used to evaluate antibacterial efficacy against a strain of bacteria commonly found in biofilms growing on the inner surface of industrial oil and gas pipelines. The bacteria used for this study was Desulfovibrio vulgaris (ATCC#29579); a Gram-negative sulfate reducing organism that has been implicated in the process of microbiologically-influenced corrosion. This organism is a facultative anaerobe, and typically grows in nature in environments that contain little or no oxygen. Thus, prior to the experiments the bacteria was grown on pre-reduced solid media (brucella blood agar enriched with haemin and vitamin K) at 37° C. under anaerobic conditions. The exposures to photodynamic disinfection were run in both anaerobic and aerobic conditions in order to determine the importance of local oxygen during the photoreaction. It is generally accepted that type II photoreactions are oxygen-dependent whereas type I photoreactions do not require oxygen, although the overall contribution of each type of photoreaction during photodynamic disinfection is not fully understood. In addition, several experimental conditions were run in the presence of L-tryptophan, which is a known scavenger of singlet oxygen and other oxygen derived free radicals. The addition of L-tryptophan was designed to test whether the antimicrobial killing effect of photodynamic disinfection was in fact dependent on the generation of reactive forms of oxygen. Overall, the experimental conditions that cultures of D. vulgaris were exposed to in this study were the following: 1) 0.01% w/v aqueous methylene blue with illumination in anaerobic environment, 2) 0.01% w/v aqueous methylene/20 mM L-tryptophan with illumination in anaerobic environment, 3) 0.01% w/v aqueous methylene blue with illumination in aerobic environment, and 4) 0.01% w/v aqueous methylene/20 mM L-tryptophan with illumination in aerobic environment. In addition, controls consisting of exposure to either photosensitizer or illumination alone were run to ensure that neither of these variables contributed to bacterial killing on their own.

For these photodynamic disinfection experiments, log phase planktonic bacterial inoculum was prepared to a concentration of 10⁷ colony-forming units per milliliter (CFU/ml) in a buffered saline solution. Samples of inoculum were then added to photosensitizer solution (aqueous methylene blue) or water (for no treatment controls) in opaque 96-well plates, giving a final sample volume of 200 microliters. Immediately after exposure to photosensitizer, bacterial samples were stirred (800 rpm using a magnetic stir bar) and illuminated with an energy dose of 20.6 Joules per square centimeter (340 milliwatts per square centimeter for 60 seconds) using a non-thermal 670 nanometer diode laser. After illumination, samples were serially diluted and plated on tryptic soy agar for 48 hours at 37° C. Raw surviving colony counts for each experimental condition were averaged and back-calculated given dilution factor to give data in CFU/ml. The data was presented as CFU/ml of surviving organisms after treatment, and kill rate was calculated as this value in experimental samples vs. control (no light, no photosensitizer) and expressed as both a log and percentage reduction in viability. The entire experimental protocol was performed in the dark, and all samples were exposed and plated one at a time to limit ambient light exposure and to maintain the exposure time of bacteria to test solution uniform across all replicates. All experimental and control conditions were run in triplicate for each individual experiment, and experiments were performed on three separate occasions to verify reproducibility. Results of exposures of D. vulgaris to photodynamic disinfection in aerobic conditions showed a powerful killing effect, with a 4.4±2.3 log₁₀ reduction in viability vs. non-treated controls (i.e. >99.99% killing). This effect was significantly reduced in the presence of L-tryptophan, where a reduction of only 1.7±0.6 log₁₀ was observed after the same exposure parameters. This result suggests that the photodynamic disinfection killing mechanism under aerobic conditions is highly dependent on type II photoreactions involving oxygen and the generation of oxygen derived free radicals. When photodynamic disinfection was performed in anaerobic conditions, the killing effect was also strong, with a 3.5±0.8 log₁₀ reduction vs. non-treated control (i.e. >99.9% killing) observed. Interestingly, however, when L-tryptophan was added and photodynamic disinfection was performed in an anaerobic environment, bacterial killing remained significant (2.5±2.1 log₁₀ reduction from non-treated control). The implications of these results are twofold: 1) photodynamic disinfection is effective in a low/no oxygen environment such as that found inside oil and gas pipelines, and 2) the mechanism of antibacterial action for photodynamic disinfection is strongly oxygen-dependent when atmospheric oxygen is present, but becomes largely oxygen-independent (i.e. type I photoreactions) in the absence of atmospheric oxygen. No significant antibacterial action was observed in control conditions consisting of either photosensitizer composition or illumination alone.

This study supports the present novel invention as it showed that photodynamic disinfection effectively eradicates a species of sulfate-reducing bacteria found in industrial oil and gas pipelines. These types of bacteria are very different, phenotypically and metabolically, from the bacteria involved in infections of humans and animals. For this reason, the present results are not obvious based on previous reports of antibacterial action using photodynamic disinfection. Furthermore, the fact the >99.9% of the bacteria were killed by photodynamic disinfection in the complete absence of oxygen, by an apparently singlet oxygen independent mechanism, suggests that this technology could be deployed in the inner pipeline environment to fight microbiologically-influenced corrosion.

### Example X

### Photodynamic Killing of Sulfate-Reducing Bacterial Biofilms in an Anaerobic Environment Using a Narrow Wavelength Laser Light Source

As corrosion-causing bacteria residing on the inner surface of pipelines are primarily present in biofilm form, it was important to test the antibacterial efficacy of photodynamic disinfection against biofilms of common sulfate reducing organisms. The study described in this example used a 670 nanometer non-thermal laser light source to illuminate biofilms exposed to the phenothiazine photosensitizer methylene blue. In addition, the same types of biofilms were exposed to the chemical disinfectant benzalkonium chloride (BAC). BAC is a cationic surfactant belonging to the quaternary ammonium group, and is one of the most common biocides currently used in chemical inhibitor formulations used to prevent internal pipeline corrosion.

Homogenous biofilms of the sulfate reducing organisms D. vulgaris (ATCC#29579) and D. desulfuricans (ATCC#14563) were grown on hard plastic pegs using a previously published protocol and system (Innovotech, Calgary, Canada). Briefly, bacterial suspensions of D. vulgaris and D. desulfuricans consisting of 10⁸ CFU/ml were prepared in modified Barrs medium for sulfate reducers (ATCC), then diluted 1:40. 200 μl of this inoculum was added to each well of a 96-well plate. 96-peg lids were then placed into the wells and the assembly was incubated at 37° C. on an Excella E2 gyrorotary shaker (Fisher Scientific) at 125 rpm for 24 hours. For both biofilm protocols, all inoculum and plates were prepared and incubated anaerobically. During the incubation period, the visible formation of an insoluble black residue in each well indicated bacterial metabolism, and after incubation a visible biofilm was evident on each pegs. Recovery experiments showed that ˜10⁶ (D. vulgaris) and ˜10⁵ (D. desulfuricans) viable bacteria per milliliter could be recovered from these biofilm pegs in the absence of any antibacterial treatment.

For treatment of biofilm pegs using photodynamic disinfection, the protocol was as follows. Using sterile forceps, pegs were broken away from the lid at the base and placed in wells of a 96-well plate in 300 μl sterile phosphate buffered saline (PBS) for 60 seconds as a rinse to remove any planktonic, free-floating cells. Pegs were then placed in 300 μl of photosensitizer solution or sterile water (controls) for 30 seconds. The pegs were subsequently either held inverted for 60 seconds in the dark as a control, or illuminated (total energy dose of 13.2 Joules) using a non-thermal 670 nanometer diode laser. Immediately after illumination, biofilm pegs were placed in 1 milliliter of pre-reduced recovery media (PBS/0.5% Tween®-80). Disruption and recovery of surviving organisms from the peg was carried out by vortexing for 10 seconds followed by a 5 minute ultrasonication (Model 250HT ultrasonicator, VWR), and finally a second vortexing for 10 seconds. After recovery, samples from the recovery medium were serially diluted and plated on brucella blood agar supplemented with haemin and vitamin K. Plates were grown anaerobically at 37° C. for 72 hours until countable colonies were present in control plates. The data was presented as CFU/ml of surviving organisms after treatment, and kill rate was calculated as this value in experimental samples vs. control (no light, no photosensitizer) and expressed as both a log and percentage reduction in viability. All experimental and control conditions were run in the dark in triplicate, and controls consisting of photosensitizer alone and illumination alone were run to verify that neither parameter influenced bacterial killing on its own.

For treatment of biofilms with benzalkonium chloride (BAC), biofilms grown on pegs as described above were used. 96-peg lids were removed from the bacterial inoculum after 24 hours of incubation, and rinsed twice to remove any free-floating organisms by placing the peg lid in 96-well plates containing 300 μl of sterile water. Pegs were then placed in aqueous solutions containing either 0% (water only control), 0.01%, 0.1%, or 1% BAC for 30 seconds. After this exposure pegs were immediately placed in recovery media and processed/plated as described above for enumeration of surviving organisms. All BAC exposure conditions were run in triplicate in anaerobic conditions for each organism.

The results of this study also supports the present novel invention as it showed that photodynamic disinfection effectively eradicates biofilms of sulfate reducing bacteria in a photosensitizer concentration dependent manner. When biofilms were exposed to 0.1% w/v methylene blue followed by laser illumination, viability of D. vulgaris was reduced by 3.2 log₁₀ (>99.9%) vs. non-treated control. Similarly, the viability of D. desulfuricans biofilms was reduced by 3.0 log₁₀ (99.9%) vs. non-treated control using the same treatment parameters. Using a lower concentration of methylene blue (0.01% w/v) kills were reduced, with a 0.5 log₁₀ (50%) reduction vs. non-treated control observed for both organisms after laser illumination. Neither the methylene blue formulation nor the laser light alone led to any significant reduction in biofilm viability for either organism tested. Biofilm exposure to the chemical disinfectant BAC yielded significantly lower kill rates than the 0.1% methylene blue photodynamic disinfection condition. Reductions in viability of D. vulgaris biofilms were 0.5 log₁₀, 1.4 log₁₀, and 1.5 log₁₀ vs. non-treated control after 30 second exposure to 0.01%, 0.1%, and 1% BAC, respectively. Reductions in viability of D. desulfuricans biofilms were 0.6 log₁₀, 2.7 log₁₀, and 1.9 log₁₀ vs. non-treated control after 30 second exposure to 0.01%, 0.1%, and 1% BAC, respectively. These results demonstrate that photodynamic disinfection using narrow wavelength laser illumination effectively eradicates biofilms of corrosion causing bacteria under anaerobic conditions, and that this killing effect is greater than that achieved with current chemical disinfection techniques under similar exposure parameters.

### Example XI

### Photodynamic Killing of Sulfate-Reducing Bacterial Biofilms in an Anaerobic Environment Using a Broadband Light Source

In this study, biofilms of sulfate reducing bacteria were treated with photodynamic disinfection as in Example X above. In these experiments, however, illumination of the biofilms was performed using a broadband (wide wavelength) light source instead of a laser. In addition, a cocktail of several different photosensitizers (rose bengal, safranin-O, methylene blue, and toluidine blue O) was tested for antibacterial efficacy along with the phenothiazines methylene blue and toluidine blue O alone.

For the broadband light photodynamic disinfection exposures, biofilms of D. vulgaris and D. sulfuricans were grown on the inner surface of flat-bottomed 96-well plates. In brief, 50 μl of liquid inoculum in log growth phase was added to each well, and plates were then incubated (37° C.) on an Excella E2 gyrorotary shaker at 125 rpm for 48 hours in order to allow biofilm growth. Recovery experiments showed that 10⁷-10⁸ viable bacteria per milliliter could be recovered from these biofilms in the absence of any antibacterial treatment. After this growth period, plates were removed from the shaker into an anaerobic cabinet, where all photodynamic disinfection treatments were performed. For treatment of biofilms on the inner surface of wells, the protocol was modified slightly from the peg treatment method described in Example X. Using a pipetter, all liquid inoculum was removed, and the well was rinsed twice with 200 μl sterile water to ensure that all free-floating planktonic organisms were removed. After the second wash, all liquid was again removed from the well and 200 μl of photosensitizer composition (or water, for no photosensitizer controls) was added for 30 seconds. At the end of this time, excess photosensitizer composition was pipetted from the well and the biofilm was illuminated for 60 seconds using a XD-301 series 150 watt haloid lamp cold light source. Immediately after illumination, recovery media was added to the well. Each biofilm well was treated separately, and all conditions were run in triplicate. After all treatments were completed, the biofilm plate was placed in an ultrasonicator for 30 minutes to release any surviving organisms into the recovery media. Samples of recovery media were then serially diluted and spot-plated on pre-reduced brucella blood agar (supplemented with haemin and vitamin K). Plates were grown anaerobically at 37° C. for 72 hours until countable colonies were present in control plates. Raw surviving colony counts for each experimental condition were averaged and back-calculated given dilution factor to give data in CFU/ml. The data was presented as CFU/ml of surviving organisms after treatment, and kill rate was calculated as this value in experimental samples vs. control (no light, no photosensitizer) and expressed as both a log and percentage reduction in viability. The entire experimental protocol was performed in the dark, and all biofilms were exposed and plated one at a time to limit ambient light exposure and to maintain the exposure/illumination time uniform across all replicates. Controls consisting of photosensitizer composition alone and illumination alone were run to verify that neither parameter influenced bacterial killing on its own.

The results of this study suggested that illumination from a broadband light source can also activate a photosensitizer composition and lead to killing of corrosion causing bacterial biofilms. Biofilms of D. vulgaris exposed to 0.1% w/v methylene blue followed by broadband illumination showed a 1.5 log₁₀ (˜95%) decrease in viability compared to non-treated control. When exposed to 0.05% w/v toluidine blue O instead of methylene blue prior to illumination, D. vulgaris biofilm viability was reduced by 1.1 log₁₀ (>90%) compared to non-treated control. Finally, when exposed to a cocktail of photosensitizers consisting of 0.05% w/v of each of methylene blue, toluidine blue O, safranin-O, and rose bengal, viability was reduced by 0.7 log₁₀ (˜70%) vs. non-treated control. When biofilms of D. desulfuricans were tested, exposure to 0.1% methylene blue followed by 60 second broadband illumination did not result in a significant reduction in viability. However, exposure to the photosensitizer cocktail described above prior to broadband illumination led to a 0.8 log₁₀ (˜80%) reduction in viability from non-treated control. Neither the photosensitizer cocktail nor the broadband light alone led to any significant reduction in biofilm viability for either organism tested. While the kills observed in this study were not as pronounced as those seen using laser illumination in Example X, using a broadband source still resulted in measurable eradication of sulfate reducing bacterial biofilms. This study supports the present novel invention as it showed that photodynamic eradication of industrial corrosion causing biofilms under anaerobic conditions using broadband light. It is likely that more powerful broadband light sources will be able to achieve even greater kills using the photosensitizers tested in this study.

What is claimed is:

1. A method to inhibit or eliminate microbes located within a pipeline comprising:
a. applying a photosensitizer composition to a predetermined interior surface area of the pipeline where microbes are located; and
b. applying light to the area at a wavelength that is absorbed by the photosensitizer composition so as to inhibit the microbes;
wherein the light is applied by a photodynamic disinfection device adapted to provide light to the area.

2. The method of claim 1 wherein the method further includes mechanically cleaning the area using cleaning means before the photosensitizer composition application step a.

3. The method of claim 1 wherein the method further includes mechanically cleaning the area using cleaning means after the light application step b.

4. The method of claim 1 wherein both the photosensitizer application step a and the light application step b are repeated at least one more time to the area.

5. The method of claim 1 wherein the photosensitizer composition contains more than one photosensitizer and each of the more than one photosensitizer exhibits a different optimal light absorbance (lambdamax).

6. The method of claim 1 wherein the photosensitizer composition contains at least one agent selected from the group consisting of dissolved atmospheric oxygen, oxygen carrying molecules capable of releasing oxygen or increasing the partial pressure of oxygen in liquid solution, molecules that can undergo reactions to yield reactive oxygen species, and a combination thereof

7. The method of claim 1 wherein the photosensitizer composition contains at least one antimicrobial agent that inhibits the microbes by a mechanism that is different from photodynamic disinfection.

8. The method of claim 1 wherein the photodynamic disinfection device is also used during the photosensitizer application step to apply the photosensitizer composition to the area.

9. The method of claim 1 wherein the microbes are selected from the group consisting of Enterobacter, Citrobacter, Eubacterium, Clostridium, Desulfovibrionales, Desulfobacterales, Syntrophobacterales, thiosulfate reducing anaerobes, tetracholoroethene degrading anaerobes, triethanolamine degrading bacteria, denitrifiers, xylan degrading bacteria, Nitrospirae, Halomonas spp., Idiomarina spp., Marinobacter aquaeolei, Thalassospira sp., Silicibacter sp., Chromohalobacter sp., Bacilli, Comamonas denitrificans, Methanobacteriales, Methanomicrobiales, Methanosarcinales, and a combination thereof.

10. A method to inhibit or eliminate microbes located within a pipeline comprising:
a. applying a photosensitizer composition comprising about 0.1% w/v methylene blue to a predetermined interior surface area of the pipeline where microbes are located; and
b. applying light to the area at a wavelength of about 670 nm that is absorbed by the photosensitizer composition so as to inhibit the microbes;
wherein energy dose provided by the light to the area is at least 1 Joules/cm²

11. A pipeline photodynamic disinfection device comprising:
a pipeline inspection gauge (pig) adapted to include a circumferential high energy light source and an energy source wherein the light source is in communication with and powered by the energy source via communication means and the circumferential high energy light source is adapted to perform photodynamic disinfection of a pipeline.

12. The device of claim 11 further comprising a cartridge adapted to contain and to dispense a photosensitizer composition onto interior surface area of the pipeline designated for photodynamic disinfection and a reservoir for collecting used photosensitizer composition.

13. The device of claim 11 further comprising cleaning means to facilitate removal of microbes from interior surface area of the pipeline.

14. The device of claim 11 wherein the circumferential high energy light source is an LED array.

15. The device of claim 12 wherein the cartridge further includes dispensing means comprising a spray mechanism to apply the photosensitizer composition at high pressure directly to the interior surface area of the pipeline designated for photodynamic disinfection treatment.

16. The device of claim 12 wherein the reservoir further includes removal means that facilitate removal and delivery of the used photosensitizer composition from the interior surface area of the pipeline designated for photodynamic disinfection treatment into the reservoir.

17. The device of claim 11 wherein the communication means is contained with an internal channel that exists between the circumferential high energy light source and the energy source.

18. The device of claim 11 wherein the energy source is comprised of a battery pack and the energy source further includes control means that allows the energy source to be controlled remotely via means selected from the group consisting of radio frequency, radioisotope thermoelectric generator, and a combination thereof.

19. The device of claim 11 wherein the pig further includes means to control speed of the device as the device travels through the pipeline.

20. The device of claim 12 wherein the device further includes at least one more of each of following components: the pig, the cleaning means, the cartridge, the reservoir; wherein the at least one additional pig has the circumferential high energy light source and the energy source.
